(19) United States
(12) Patent Application Publication
(10) Pub. No.: US 2004/0115233 A1
(43) Pub. Date: Jun. 17, 2004

(54) Composition for improving skin environment and clothes thereof

(76) Inventors: Katsumori Fukumoto, Wakayama (JP); Takanobu Ono, Kanagawa (JP); Youichi Fukumoto, Tokyo (JP)

Correspondence Address: Wenderoth Lind & Ponack, Suite 800, 2033 K Street NW, Washington, DC, 20006, US

(21) Appl. No.: 10/474,797

(22) PCT Filed: Apr. 12, 2002
(86) PCT No.: PCT/JP02/03702

(30) Foreign Application Priority Data
Apr. 12, 2001 (JP) 2001-113621

(51) Int. Cl. A61K 7/075; A61K 7/08; A61K 7/06; A61K 9/70

(52) U.S. Cl. 424/401; 424/74; 424/70.22; 424/443

(57) ABSTRACT

The skin condition-improving composition of the invention comprises any one of tea tree oil and lemon-scented tea tree oil, and γ-linolenic acid, and provides an environment capable of curing atopic dermatitis and asteatosis, and suppressing itching.

### TECHNICAL FIELD

The invention relates to a skin condition-improving composition and clothes processed therewith.

### BACKGROUND ART

Considerable attention has been given to reexamination and reevaluation of the effectiveness of non-steroidal agents as external medicines (externally applied medicines) for atopic dermatitis. Non-steroidal external medicines have often been used for mild atopic dermatitis. Dr. Nobuo Yamamoto stated that external non-steroidal medicines are used principally for the treatment of faces that are liable to suffer side effects from external steroidal medicines, and when mild inflammation appears (see “Guidelines for Treatment of Atopic Dermatitis”, allotted research by Nobuo Yamamoto, Kousei Kagaku Kenkyu, 1999). Since non-steroidal medicines have a weak anti-inflammatory action compared with steroidal medicines and can induce contact dermatitis, their applicable range has actually been considered to be narrow.

Only the external steroidal medicines are able to sufficiently suppress inflammatory symptoms of atopic dermatitis and have been being proved to be effective and safe on scientific bases. On the other hand, most patients reported in the literature who showed an effective response to treatment with non-steroidal external medicines were mild cases. Mild atopic dermatitis may be sufficiently controlled with humectants, and external non-steroidal medicines are not always considered necessary. According to this idea, only external steroidal medicines are capable of promptly and reliably depressing inflammation in atopic dermatitis, and to alleviating pains in patients. See “Atopic Dermatitis, Chapter 2, Management and Care, First Choice Therapy, Section 3, External Therapy (1), Principle of External Therapy”, edited by Yoshiki Miyaji (Professor of Dermatology, Medical Department, Kyoto University) and Toshikazu Nagakura (Director of Yoga Allergy Clinic); published by Hideaki Matsuoka, Medical Review Co.; publishing date Apr. 10, 2000;

In addition to the above, γ-linolenic acid, which is contained in evening primrose (Japanese name Tukimiso) including Ohmatsuyoigusa (Japanese name; scientific name Oenothera erythrosepala Borbas) and Matsuyoigusa (Japanese name; scientific name Oenothera odorata Jacq.), both common in Japan, is used for the therapy of patients with atopic diseases.

Dr. David Horrobin (Professor of Montreal University), a worldwide authority on unsaturated fatty acid researches, has developed an atopic disease medicine called “GLA” using γ-linolenic acid, and has proposed a novel therapeutic method involving the oral administration of evening primrose seed oil.

It was revealed that the concentration of γ-linolenic acid in the blood of patients with atopic dermatitis is only 50% of that in the blood of normal persons, and that the concentration of γ-linolenic acid in the milk of mothers with atopic conditions is also 50% or less than that of normal mothers. Accordingly, Dr. David Horrobin has identified the evening primrose as a γ-linolenic acid supplement from plants growing in nature. He has succeeded in extracting γ-linolenic acid contained in the seeds of the evening primrose.

The effectiveness of γ-linolenic acid for the therapy of the atopic diseases has also been proved in a clinical trial performed at Bristol University. γ-Linolenic acid was administered to patients with mild to medium atopic diseases (including 127 children and 240 adults), and it was reported that the medicine is effective for alleviating itching. The disease was improved in 116 of 179 serious cases in the trial (C. R. Lovell, J. B. Burton and D. F. Horrobin, The Lancet, Jan. 31, 1981; see the home page of Japan Functional Food Research Association, http://www.jafra.gr.jp/ganma.htm).

Although the effectiveness of oral administration of γ-linolenic acid has been reported, the uses of γ-linolenic acid as an external medicine have been disregarded in recent years. This is because effectiveness of steroids as external medicines has been emphasized as described above, and effectiveness of non-steroidal medicines as external medicines has been considered doubtful.

Accordingly, the concept of developing underwear and *skin wear containing γ-linolenic acid for patients suffering from dermatitis as well as from asteatosis has not previously been considered, that is, its use as an external medicine has not been realized.

### DISCLOSURE OF INVENTION

The inventors have devised a method of utilizing γ-linolenic acid to improve the skin conditions, particularly the skin condition against atopic dermatitis and asteatosis, and achieved the invention through intensive studies in which γ-linolenic acid or α-linolenic acid, tea tree oil and lemon-scented tea tree oil were found to be effective for improving the skin condition. The inventors also found a method for continuously supplying these compositions to the skin through underwear.

In a first aspect, the invention provides a skin condition-improving composition containing any one of γ-linolenic acid and α-linolenic acid.

In a second aspect, the invention provides a skin condition-improving composition containing a bacteriostatic agent and any one of γ-linolenic acid and α-linolenic acid.

In a third aspect, the invention provides a skin condition-improving composition containing any one of terpinene-4-ol, citral and citronellal, and any one of γ-linolenic acid and α-linolenic acid.

In a fourth aspect, the invention provides clothes comprising at least any one of γ-linolenic acid, α-linolenic acid, tea tree oil and lemon-scented tea tree oil fixed on the clothes.

In a fifth aspect, the invention provides clothes comprising any one of γ-linolenic acid, α-linolenic acid, tea tree oil and lemon-scented tea tree oil fixed on the clothes as a clathrate compound with monochlorotriazinyl-β-cyclodextrin.

In a sixth aspect, the invention provides a skin condition-improving composition comprising any one of γ-linolenic acid and α-linolenic acid, and a bacteriostatic agent, at least one of which is formed into a clathrate compound with monochlorotriazinyl-β-cyclodextrin.

The skin condition-improving composition as used herein refers to a composition for providing clothes worn on the body with pharmaceutical effects against skin diseases such as atopic dermatitis and asteatosis.

The clothes as used herein not only refers to usual clothes and apparel, but also to night clothes, underwear, skin wear, bath robes, gowns, girdles, cooking aprons, swimwear, sportswear and bedclothes (bed sheets and pillow covers) as well scarves, masks, socks, gloves, hats or caps, dressings, and supporters.

The skin condition-improving composition according to the first aspect of the invention contains γ-linolenic acid or α-linolenic acid as an effective agent proven in recent years to be effective in oral administration therapy of atopic dermatitis. However, the composition of the invention is not orally administered. Instead, the above composition is used to improve the environment around the skin where symptoms of atopic dermatitis and asteatosis have developed. In other words, the composition according to the first aspect of the invention can realize an environment that enables atopic dermatitis and asteatosis to be cured by employing the above invention.

The skin condition-improving composition according to the second aspect of the invention contains γ-linolenic acid or α-linolenic acid as well as a bacteriostatic agent that is effective at suppressing the growth of microorganisms on fibers and alleviating itching, thereby realizing an environment that enables therapy for dermatitis and asteatosis and alleviation of itching. The bacteriostatic agent as used herein refers to non-steroidal agents having disinfectant effects and microbial growth-suppressing effects such as tea tree oil and lemon-scented tea tree oil.

The skin condition-improving composition according to the third aspect of the invention enables the function of the skin condition-improving composition according to the second aspect of the invention to be more effectively manifested by containing terpinene-4-ol as a particularly effective component of tea tree oil, or citral or citronellal as a particularly effective component of lemon-scented tea tree oil, together with γ-linolenic acid.

In the fourth aspect of the invention, the environment around the affected part of the skin is improved since the underwear and underclothes that directly contact the skin contain at least one of γ-linolenic acid, α-linolenic acid, tea tree oil and lemon-scented tea tree oil, thereby enabling atopic dermatitis and asteatosis to be more efficiently cured.

In the fourth aspect, the surrounding environment such as underwear and underclothes, which are always in contact with the skin manifesting atopic dermatitis and asteatosis, is improved rather than attempting to treat the affected parts by oral administration of the medicine to the patient, thereby enabling rashes, chapping and itching due to atopic dermatitis and asteatosis to be suppressed.

In the fifth aspect of the invention, the composition achieves excellent resistance to washing, thus enabling the effect in the fourth aspect of the invention to be maintained for a long period of time.

In the sixth aspect, the invention provides a skin condition-improving composition comprising any one of γ-linolenic acid and α-linolenic acid, and a bacteriostatic agent, and at least one of them is clathrated with monochlorotriazinyl-β-cyclodextrin.

γ-Linolenic acid is also named 6,9,12-octadecatrienic acid, and is represented by the following chemical formula:

CH₃ (CH₂)₄CH═CHCH₂CH═CHCH₂CH═(CH₂)₄COOH

The compound is a linear triene fatty acid with a carbon number of 18 having cis-double bonds at positions 6, 9 and 12, and is represented by the molecular formula of C₁₈H₃₀O₂ with a molecular weight of 278.43.

A large quantity of γ-linolenic acid is contained in Primulaceae (Japanese name Sakurasou; Primula sieboldii E. Morren) seed oil as well as in other plants, and 6,9,12,15-octadecatetraenoic acid having an additional unsaturated bond at the methyl terminal is also contained in the plants. A trace amount of the compound is also found in animal tissues. Linolenoyl-CoA is derived from γ-linoloyl-CoA by the enzyme system in the endoplasmic reticulum membrane of the liver, and is further converted into 8,11,14-eicosatrienoyl-CoA by chain elongation followed by conversion into arachidonic acid by unsaturation at position 5.

α-Linolenic acid is contained in soy bean oil, rape seed oil and Lamiaceae (Japanese name Siso) oil, and is represented by the following chemical formula:

CH₃ (CH₂CH═CH₃) 3 (CH₂)₇COOH

Tea tree oil as used herein refers to an essential oil obtained by distillation of Melaleuca alternifolia oil.

The tea tree is a small tree belonging to the Eucalyptus family, and tea tree oil is an essential oil obtained by distillation of extracts from the tea tree oil. The scientific name of the tea tree is Melaleuca alternifolia, a small tree growing in nature in restricted regions of the seashore in the north-west part of New South Wales State, Australia. An oil having a therapeutic effect is collected mainly from the leaves of the tree. For example, the tea tree oil is an aromatic pale-yellow essential oil obtained by steam distillation of fresh leaf tips of Melaleuca alternifolia. While more than 50 genera of genus Melaleuca are known today, only the oil collected from the leaves of Melaleuca alternifolia is the object of this investigation.

The chemical composition of the tea tree oil will be described hereinafter.

The proportion of the tea tree oil obtained by distillation of the tea tree leaves is about 1.8%. While the oil contains 48 or more components, it is mainly composed of terpenes such as 1-terpinene-4-ol, γ-terpinene and p-cymene. In the Australian Standard (AS-2782-1985), so-called melaleuca oil (terpinene-4-ol type) is quoted to contain 30% or more of terpinene-4-ol and 15% or less of 1,8-cineol.

The tea tree oil mainly comprises monoterpenes, terpene alcohols and sesquiterpene, and it has been confirmed that the tea tree oil contains 96 components. Although the content of each component is irregular and not constant depending on the collected season and location of the tea tree, it is generally as shown in Table 1 below. Table 1 lists the averaged composition of the tea tree oil.

The component having the highest antibacterial action among them is terpinene-4-ol. However, paracimene, α-terpinene, γ-terpinene and α-terpineol also exhibit some antibacterial actions. The antibacterial actions are different depending on the kind of microorganisms. For example, an increase in the content of terpinene-4-ol within the range of 35% enables an antibacterial action against Pseudomonas aeruginosa, Candida albicans, Staphylococcus aureus and Aspergilus niger to be increased. On the other hand, an increase in α-terpineol enables an antibacterial action against Escherichia coli to be increased.

Only the quantities of 1,8-cineol and terpiene-4-ol are quoted in the Australian Standard of the tea tree oil.

While the tea tree oil is usually used for the treatments of external injuries as an external disinfectant, the inventors have found that the antibacterial action thereof can suppress the proliferation of bacteria that causes itching. Accordingly, the tea tree oil in the composition and clothes is at least responsible for suppressing itching according to the invention.

The lemon-scented tea tree oil is another essential oil collected from a tree called Leptospermun petersonii, a different species from Melaleuca alternifolia from which the usual tea tree oil is collected. While the ordinary tea tree oil has a eucalyptus oil-like refreshing aroma, the lemori-scented tree oil smells like a lemon. Since the shape of this tree resembles a tea tree, the oil is called lemon-scented tea tree oil, that is, “tea tree oil having lemon-like scent”.

The principal components of the lemon-scented tea tree oil comprise citral and citronellal, and the two essential oil components account for 70% or more oil's composition. Therefore, the lemon-scented tea tree oil has a different composition to the composition of the usual tea tree oil containing terpinenne-4-ol as a principal component.

The antibacterial action of the lemon-scented tea tree oil is stronger than the usual tea tree oil. Air Green Co. (1-2-40 Atobe-Honmachi, Yao City, Osaka Prefecture, Japan) found that the oil has a particularly strong antibacterial action against Escherichia coli and Staphylococcus aureus.

The lemon-scented tea tree oil is often used after blending with the usual tea tree oil in order to enhance the antibacterial action and change the aroma. Since the lemon-scented tea tree oil has a particularly strong antibacterial action, it can function as a disinfectant against methicillin-resistant Staphylococcus aureus. Antibiotics kill a bacteria by inhibiting cell wall synthesis of the bacteria. Since the action of the antibiotics is based on chemical reactions, the emergence of antibiotics resistant bacteria is inevitable. However, since the tea tree oil and lemon-scented tea three oil kill the bacteria by their own physical actions, no antibiotics-resistant bacteria will emerge. Although the mechanisms of the action have not been fully elucidated, it may be conjectured that respiration and movement of the bacteria are inhibited by surrounding the bacteria in oil films. Therefore, antibiotic-resistant bacteria are prevented from emerging. This is an important feature of the tea three oil and lemon-scented tea tree oil.

While the lemon-scented tea tree oil has been used as a spice for foods for a long period of time, its antibacterial action was first noticed in 1997, and planting of the trees was started the following year. Since at least three years' cultivation is necessary before the oil can be collected, commercial production of the lemon-scented tea tree oil has only been possible since the year 2000.

However, the drawback of lemon-scented tea tree oil is that it is susceptible to oxidation, and thus the production scale is small. Its price is higher than the usual tea tree oil. Although the price problem may be solved as the production scale increases, tea tree oil is advantageous over the lemon-scented tea tree oil with respect to price.

The lemon-scented tea tree oil is susceptible to oxidation because it contains readily oxidizable aldehyde groups in the citral and citronellal, which are major terpenoids components of the lemon-scented tea tree oil. However, Air Green Co. (1-2-40 Atobe-Honmachi, Yao City, Osaka Prefecture, Japan) have elucidated that oxidation may be prevented by blending a small quantity of vitamin E with the oil.

The components of the lemon-scented tea tree oil will be described hereinafter.

The major components of the lemon-scented tea tree oil are citral and citronellal, which account for 70% or more of the terpenoids contained in the lemon-scented tea tree oil. The composition is different from the usual tea tree oil having terpinene-4-ol as a principal component.

Table 2 shows the components of the commonly used lemon-scented tea tree oil.

γ-Linolenic acid considered to be effective to atopic dermatitis and asteatosis, and tea tree oil or lemon-scented tea tree oil are fixed to cellulose fibers of the clothes of the invention so that the chemicals are resistant to washing. The disease conditions are expected to be improved when patients with atopic dermatitis wear the processed fabrics. While the same effect may be expected by using α-linolenic acid in place of γ-linolenic acid, γ-linolenic acid will be described herein as an example of both linolenic acids.

γ-Linolenic acid and tea tree oil or lemon-scented tea tree oil are clathrated with a cyclic oligosaccharide (composed of glucose). The clathrate compound has OH (negatively charged hydroxyl) groups at the terminal.

The cyclic oligosaccharide as the clathrating compound of γ-linolenic acid and tea tree oil or lemon-scented tea tree oil is able to be tightly adhered on the cellulose fibers by covalent bonds, ionic bonds or vas der Waals' force.

The method for fixing γ-linolenic acid and tea tree oil on the fibers (clothes) will be briefly described below.

(First Fixing Method)

γ-Linolenic acid and tea tree oil are clathrated with a cyclic oligosaccharide (composed of glucose) that is able to be covalently bonded to the hydroxyl groups and amino groups, and the clathrate compound is allowed to react with the OH groups of the cellulose fiber using chlorine (Cl) to permit the clathrate compound to be resistive to washing.

(Second Fixing Method)

γ-Linolenic acid and tea tree oil are clathrated with a cyclic oligosaccharide (composed of glucose), and the clathrate compound is bonded to the fiber by ionic bonds using a softening agent having amino groups (positively charged). The clathrate compound is allowed to be resistant to washing by ionic bonds between the anionic groups of the cellulose fibers and cationic groups of the softening agent having amino groups.

(Third fixing method)

γ-Linolenic acid and tea tree oil are clathrated with a cyclic oligosaccharide (composed of glucose) or enveloped into microcapsules, and the clathrate compound or microcapsules are tightly adhered onto the fiber using a resin to permit the chemicals to be resistant to washing.

Any one of the first to third fixing methods may be employed for fixing the lemon-scented tea tree oil to the clothes together with γ-linolenic acid in place of the lemon tree oil.

γ-linolenic acid may be fixed to the clothes independently from the tea tree oil or lemon-scented tea tree oil. The same effect may be expected by using α-linolenic acid in place of γ-linolenic acid the fixing methods as described above.

β-cyclodextrin is preferably used as the oligosaccharide, and monochlorotriazinyl-β-cyclodextrin is particularly preferable.

Monochlorotriazinyl-β-cyclodextrin is a reactive cyclodextrin (CD) derivative that is able to react with the hydroxyl group and amino group with covalent bonds. The CD derivative has a solubility in water of as high as 30% or more, and the degree of substitution (DS) of the monochlorotriazinyl (MCT) groups is 4 groups (DS=0.4) per one glucose unit.

The structure is represented as STRUCTURE 1 as bellow.

The concept of adsorption (absorption) of γ-linolenic acid and tea tree oil clathrated with monochlorotriazinyl-β-cyclodextrin to the cellulose fiber will be described below. As shown in FIG. 11, γ-linolenic acid or tea tree oil as an clathrated compound a is enveloped with monochlorotriazinyl-β-cyclodextrin as an clathrating compound b. The letter C in the drawing denotes covalent bonds. The letter C represents the covalent bond of oxygen atoms in monochlorotriazinyl-β-cyclodextrin, and the covalent bonds between O⁻—Na⁺.

The chemical d is adsorbed on the cellulose fiber in the clothes (underwear) as shown in FIG. 12 (a part of the chemical d is clathrated γ-linolenic acid, and the others are clathrated tea tree oil). γ-Linolenic acid or tea tree oil as the clathrated compound a is gradually discharged in a long period of time. Discharged γ-linolenic acid and tea tree oil is adsorbed through the skin.

An example of the method for producing the clothes of the invention on which γ-linolenic acid and tea tree oil are fixed by employing the first fixing method will be described in detail with reference to the drawings.

FIGS. 1 and 2 show an example of the method for producing the clothes according to the invention (FIG. 1 shows a block diagram of the method, FIG. 2(A) illustrates a side view of a heating device used for a low temperature heating step and high temperature heating step in FIG. 1, FIG. 2(B) illustrates a front view of the apparatus shown in FIG. 2(A), and FIG. 2(C) illustrates a plane view of the apparatus shown in FIG. 2(A)).

In the method for producing the clothes of the invention, γ-linolenic acid and tea tree oil (named as a composition hereinafter) is permeated into and fixed on the textile products after a sewing process followed by dehydration, and the product is disposed in the space by, for example, being hung on a hanger for drying with hot air.

The steps of this production process will be sequentially described below.

As shown in FIG. 1, the clothes after sewing is subjected to a composition adhering step 1, low temperature heating step 2, high temperature heating step 3 and finish step 4 in this order in the method for fixing the composition.

The composition adhering step 1 comprises a primary immersion step 100 and primary wringing step 110 after the primary immersion step 100.

In the primary immersion step 100, the clothes are dipped in an immersion vessel (not shown) filled with the composition solution to allow the composition to permeate into the clothes.

In the primary wringing step 110 after the primary immersion step 100, the clothes taken out of the immersion vessel are dehydrated by wringing with a wringing machine.

The wringing machine comprises a vessel for housing the clothes, and a device having a dehydration mechanism for dehydrating the clothes by rotation of the vessel (not shown), as in the well-known dehydrator of a home-use washing machine. An excess amount of the composition permeated in the clothes are removed with the wringing machine in the primary wringing step 110.

The excess amount of the composition is removed from the clothes in which the composition has permeated in the primary wringing step 110. The composition is also facilitated to be fixed on the clothes by tightly wringing the clothes in the primary wringing step 110.

As described above, the clothes are dehydrated in the wringing step such as the primary wringing step 110.

The solution of the compound used in the primary immersion step 100 in the bacteriostatic and antibiotic agent adhering step 1 contains a clathrate compound of γ-linolenic acid (or α-linolenic acid) and tea tree oil clathrated with a cyclic oligosaccharide (composed of sucrose) having covalent bond forming reactive groups with the hydroxyl and amino groups.

The clothes after the composition adhering step 1 is subjected to heat treatment steps in the low temperature heating step 2 and high temperature heating step 3.

FIG. 2 illustrates a heating device 20 used in the low temperature heating step 2 and high temperature heating step 3.

As shown in FIGS. 2(A) and 2(B), the heating device 20 comprises a low temperature heating device 21 for the low temperature heating step 2, a high temperature heating device 31 for the high temperature heating step 3, and conveyers 30 provided in the low temperature heating device 21 and high temperature heating device 31, respectively.

The low temperature heating device 21 comprises a low temperature heating chamber 22 forming a convey passageway of the clothes within it, a steam heater 23 formed independently from the low temperature heating chamber 22, an inlet tube 24 disposed between the steam heater 23 and low temperature heating chamber 22 for connecting between them (FIGS. 2(A), 2(B) and 2(C)), and a discharge duct 25 disposed between the steam heater 23 and the lower side wall of the low temperature heating chamber 22 for connecting between them (FIG. 2(C)).

The low temperature heating chamber 22 is a housing having an width L1 of about 12.4 m and a height of about 2.5 m.

The steam heater 23 is able to generate about 30,000 kcal of heat using steam, and hot air is sent into the low temperature heating chamber 22 through the inlet tube 24 as shown in FIG. 2(A). The inlet tube 24 is branched into a plurality of branch tubes 24a at the low temperature heating chamber 22 side. Each of the branch tubes 24a is connected to the upper part of the low temperature heating chamber 22, and hot air is introduced into the low temperature heating chamber 22 through the branch tubes 24a.

Hot air introduced into the low temperature heating chamber 22 is discharged from the low temperature heating chamber 22 toward the steam heater 23 through the discharge duct 25.

As shown in FIG. 2(B), an inlet 22a for introducing the clothes into the low temperature heating chamber 22 is formed at the front face of the low temperature heating chamber 22. An air nozzle 22b is provided at the upper part of the inlet 22a. The air nozzle 22b is provided for closing the inlet 22a by forming an air curtain by blowing air down. The inside of the low temperature heating chamber 22 is isolated from the atmosphere by means of this air curtain.

The high temperature heating device 31 comprises a high temperature heating chamber 32 provided by being elongated at the rear part of the low temperature heating chamber 22 of the low temperature heating device 21 so that a passageway for conveying the clothes is formed within it, an air heater 33 formed independently from the high temperature heating chamber 32, an inlet tube 34 disposed between the air heater 33 and high temperature heating chamber 32 for connecting between them, and a discharge duct 35 disposed between the air heater 33 and lower side wall of the high temperature heating chamber 32 for connecting between them (FIG. 2(C)).

The high temperature heating chamber 32 is formed by being integrated with the low temperature heating chamber 22, and has an width L2 of about 6.2 m. The height of the high temperature heating chamber 32 is the same as the height of the low temperature heating chamber 22.

The air heater 33 is able to generate hot air at 200° C. using light petroleum oil as a fuel, and generated hot air is sent into the high temperature heating chamber 32 through the inlet tube 34. The inlet tube 34 is branched into a plurality of branch tubes 34a at the high temperature heating chamber 32 side as shown in FIG. 2(A). Each of the branch tubes 34a is connected to the upper part of the high temperature heating chamber 32, and hot air is introduced into the high temperature heating chamber 32 through the branch tubes 34a.

Hot air introduced into the high temperature heating chamber 32 is discharged from the high temperature heating chamber 32 toward the air heater 33 through the discharge duct 35.

As shown in FIG. 2(B), an outlet 32a for carrying the clothes out of the high temperature heating chamber 32 is provided at the back face of the high temperature heating chamber 32. An air nozzle 32b is provided at the upper part of the outlet 32a. The outlet 32a is closed by forming an air curtain by blowing air down from the nozzle 32b. The inside of the high temperature heating chamber 32 is isolated from the atmosphere by means of this air curtain.

The conveyer 30 comprises a conveyer chain 30a capable of suspending a plurality of hangers, and a holding driving member 30b that permits the conveyer chain 30a to circulate.

The holding driving member 30b holds the conveyer chain 30a in order to permit the conveyer chain 30a to circulate. The conveyer chain 30a is provided to form a circle, and travels from the inlet 32a of the low temperature heating chamber 22 into the low temperature heating chamber 22, and from the outlet 32a of the high temperature heating chamber 32 to the outside through the inside of the high temperature heating chamber 32. The conveyer chain arrives the inlet 22a of the low temperature heating chamber 22 again after passing through the upper parts of the housings 22 and 32. The conveyer chain 30a circulates the passageway as described above.

A conveyer screw bar may be used in place of the conveyer chain 30a. An external thread is formed at the outer circumference of the conveyer screw bar, and the hangers are sent forward by allowing the screw bar to turn in the advancing direction while suspending the hangers. The hangers are allowed to travel backward by inversely turning the screw bar. The conveyer chain in the descriptions hereinafter may be changed to the conveyer screw bar, and both conveyer means may be used in combination.

The clothes after the composition adhering step 1 are sequentially hung on the hangers, and are suspended under the conveyer chain 30a at a position in front of the inlet 22a of the low temperature heating chamber 22 of the heating device 20.

The clothes hung on the hangers and suspended under the conveyer chain 30a are advanced toward the inside of the low temperature heating chamber 22 and high temperature heating chamber 32 in the direction of advance. While the materials of the hangers used for hanging the clothes may be selected with the proviso that they are resistant to the heating temperature, the hungers made of stainless steel are suitable. The shape of the hanger may be the same as used in a home and laundry.

The clothes hung on the hangers are dried by heating by allowing the hangers to advance into the low temperature heating chamber 22 by means of the conveyer chain 30a in the low temperature heating step 2.

The clothes are dried by heating at a temperature of 70 to 80° C. (the temperature in the low temperature heating chamber 22) in the low temperature heating chamber 22 with hot air from the steam heater 23. The clothes hung on the hangers are transferred into the high temperature heating chamber 32 after traveling in the low temperature heating chamber 22 kept at the temperature above for about 10 minutes. The heating temperature and heating time are variable depending on the materials of the clothes to be processed.

The clothes transferred from the low temperature heating chamber 22 is dried with hot air in the high temperature heating step 3 by allowing the clothes to advance into the high temperature heating chamber 32 by means of the conveyer chain 30a.

The clothes are dried with hot air from the air heater 33 in the high temperature heating chamber 32 for 2 to 8 minutes at a temperature of 120 to 180° C. (the temperature in the high temperature heating chamber 32). The clothes hung on the hangers are conveyed to the outside from the outlet 32a after allowing the clothes to travel in the high temperature heating chamber 32 kept at the temperature above for 2 to 8 minutes. The heating temperature and time are also variable depending on the materials of the clothes to be treated.

It is desirable that the clothes are heated with hot air for 2 to 8 minutes at 120 to 180° C. in the high temperature heating step 3 in order to enhance the fixing ratio of the composition onto the fibers of the clothes, although the conditions differ depending on the materials and quality of the clothes.

Since the clothes are conveyed in the heating device 20 while they are hung on the hangers in the drying steps 2 and 3, the entire surface of the clothes is uniformly exposed to hot air while enabling the excess solution of the composition to be vertically dripped. Accordingly, the clothes may be dried without allowing the distribution of the composition to be uneven. Since the surfaces of the clothes do not contact other surfaces with no uneven distribution of the composition, the composition may be evenly adhered on the clothes.

The finish step 4 comprises a smoothening step 400 and wrapping step 410 after the smoothening step 400.

In the smoothening step 400, the clothes after the low temperature heating step 2 and high temperature heating step 3 is taken out of the conveyer chain 30a, and is smoothened with a steam iron.

In the wrapping step 410, the clothes are folded and packed in bags followed by hanging on the hangers.

The entire steps are completed after the finish step 4.

The fixing ratio of the composition is markedly improved by applying the high temperature heating step 3 after low temperature heating in the low temperature heating step 2.

The fixing ratio of the clothes after the treatment can be improved by drying the clothes by hanging on the hangers in the high temperature heating step 3.

While the composition was adhered on the clothes by directly immersing the clothes in the composition, the composition may be sprayed onto the clothes. However, a better fixing ratio may be obtained by the method for wringing the clothes after immersion than the spray method above. Accordingly, the wringing method after immersion is desirable in the invention considering the fixing ratio.

The chemicals adhered on the clothes are not restricted to the skin condition-improving composition for atopic dermatitis, and other chemicals having bacteriostatic and antibacterial properties as well as various effects such as water repelling, insect repelling, flame retarding, aromatic, antistatic and shape retaining properties may be used together.

In the primary wringing step 110 in the composition adhering step 1, a device for wringing the clothes (not shown) by passing the clothes through two rollers arranged in series may be used in place of the device as described above.

While the clothes have been described as the treating object, the invention is applicable to any sewed fabrics other than the clothes so long as they are able to be hung on a hanger. For example, the invention is applicable to towels, bed sheets and other bedclothes, handkerchiefs, scarves, car sheets, pillow covers, table clothes, kitchingen clothes as well as cloth shoes, cloth bags, physician's clothes, surgeon's clothes, nurse's clothes, sleeping bags, and rain gears such as rain coats.

Another embodiment of the heating device 20 will be described with reference to FIG. 3. FIG. 3(A) illustrates the side view of the low temperature heating device 21 of the heating device 20, and FIG. 3(B) illustrates the side view of the high temperature heating device 31.

The heating device 20 is different from the heating device shown in FIG. 2 in that a low temperature heating device 21 are separately constructed from a high temperature heating device 31.

As shown in FIG. 3(A), the heating chamber 22 of the low temperature heating device 21 has a rear part having an inlet 22c provided thereto. An air nozzle 22d for isolating the inside of a housing 22 from the atmosphere with an air curtain is provided at the upper part of the outlet 22c. The low temperature heating device 21 comprises a conveyer 30 similar to the conveyer in FIG. 2. The conveyer 30 comprises a conveyer chain 30a capable of suspending a plurality of hangers, and a holding driving member 30b for allowing the conveyer chain 30a to circulate. The holding driving member 30b holds the conveyer chain 30a, and permits the conveyer chain 30a to circulate. The conveyer chain 30a of the conveyer 30 is formed into a circle, travels from the inlet 22a of the heating chamber 22 into the heating chamber 22, and moves to the outside from the outlet 22c. Then, the conveyer chain arrives at the inlet 22a of the heating chamber 22 through the upper part of the heating chamber 22. The conveyer chain 30a is circulated through the passageway as described above.

The low temperature heating device 21 differs from the low temperature heating device 21 shown in FIG. 2 in that it is independently constructed from the high temperature heating device 31. The heating chamber 22 of the low temperature heating device 21 has the rear part, the outlet 22c and air nozzle 22d are provided at the rear part, and the conveyer chain 30a only circulates in the heating chamber 22 of the low temperature heating device 21. However, the other constructions of the low temperature heating device 21 shown in FIG. 3(A) are the same as those of the low temperature heating device 21 shown in FIG. 2.

The high temperature heating device 31 independently provided from the low temperature heating device 21 is shown in FIG. 3(B). As shown in FIG. 3(B), the heating chamber 32 of the high temperature heating device 31 has a front part, and an inlet 32c is provided at the front part. An air nozzle 32d for isolating the inside of the housing 32 from the atmosphere with an air curtain is provided at the upper part of the inlet 32c.

The high temperature heating device 31 is provided with a conveyer 36 different from the conveyer 30 provided at the low temperature heating device 21. The conveyer 36 comprises a conveyer chain 36a capable of suspending a plurality of hangers as the conveyer 30 provided at the low temperature heating device 21, and a holding driving member 36b for permitting the conveyer chain 36a to circulate. The holding driving member 36b holds the conveyer chain 36a, and permits the conveyer chain 36a to circulate. The conveyer chain 36a of the conveyer 36 is formed into a circle, travels from the inlet 32c of the heating chamber 32 into the heating chamber 32, and moves to the outside from the outlet 32a. Then, the conveyer chain arrives at the inlet 32c of the heating chamber 32 through the upper part of the heating chamber 32. The conveyer chain 36a is circulated through the passageway as described above.

The high temperature heating device 31 is independently constructed from the low temperature heating device 21 as described above. The high temperature heating device 31 differs from the high temperature heating device 31 shown in FIG. 2 in that the heating chamber 32 of the high temperature heating device 31 comprises the front part, the inlet 32c and the air nozzle 32d are provided at the front part, the conveyer 36 is independently provided, and the conveyer chain 36a of the conveyer 36 only circulates in the heating chamber 32 of the high temperature heating device 21. However, the high temperature heating device 31 shown in FIG. 3(A) is the same as the high temperature heating device 31 shown in FIG. 2 with respect to other constructions.

The hangers of the clothes coming out of the low temperature heating device 21 are once removed from the conveyer chain 30a, and are suspended under the conveyer chain 36a after being transferred to the high temperature heating device 31.

Employing the heating device 20 comprising the separately provided the low temperature heating device 21 and high temperature heating device 31 permits the installation space to be reduced. For example, since the low temperature heating device 21 and high temperature heating device 31 are integrated into one unit in the heating device 20 shown in FIG. 2, a linear space with a longitudinal with of 18.6 m is necessary as a combined length of L1 and L2. On the contrary, the low temperature heating device 21 and high temperature heating device 31 can be disposed at separate sites with each other in the heating device 20 shown in FIG. 3. Therefore, the linear space of as long as 18 m or more is not needed.

However, the hangers is not required to be removed from the conveyer 30 for transferring to the high temperature heating step 3 in the high temperature heating device 31 after completing the low temperature heating step 2 in the low temperature heating device 21, when the heating device 20 shown in FIG. 2 is used. Consequently, the time for suspending the hangers again is saved. Otherwise, the conveyer may be continuously disposed to the low temperature heating device 21 and high temperature heating device 31 while separately providing these devices 21 and 31.

Another method for producing the clothes according to the invention will be described with reference to FIG. 4. FIG. 4 shows a block diagram of this process.

In this production method of the clothes shown in FIG. 4, a composition adhering step 1, high temperature heating step 3 and finish step 4 are applied in this order.

The composition adhering step 1 comprises a primary immersion step 100, and a wringing step 110 applied after the primary immersion step 100.

In the primary immersion step 100, the clothes are dipped in a dipping vessel (not shown) filled with the composition to allow the composition to permeate in the clothes as the method shown in FIG. 1. In the primary wringing step 110, the clothes taken out of the immersion vessel is wrung using a wringing device after the primary immersion step 100 as in the embodiment shown in FIG. 1. The same wringing device as used in FIG. 1 is also used in this process. The excess solution of the composition is removed from the clothes in which the solution of the composition has permeated in the primary wringing step 110, and fixing of the composition on the fibers is accelerated. It is preferable in this process that the weight of the clothes increases twice as large as the weight of the clothes before the composition adhesion step 1 when the primary wringing step 110 is completed.

The clothes are directly transferred to the high temperature heating step 3 after the composition adhesion step 1 without passing through the low temperature heating step 2 in the method shown in FIG. 4. The high temperature heating step 3 and the finish step 4 thereafter are the same as the processing steps in FIG. 1. The high temperature heating step 3 may be properly performed using the device shown in FIG. 3(B).

The process shown in FIG. 4 is the same as the process shown in FIG. 1, except that the composition adhesion step 1 is completed only by the primary immersion step 100 and primary wringing step 110, and that the high temperature heating step 3 is not provided.

While it is possible to omit the low temperature heating step 2 as in the process shown in FIG. 4, the processing time in the high temperature heating step 3 may be shortened by passing through the low temperature heating step 2 as in the process shown in FIG. 1. Consequently, this process is advantageous with respect to the processing cost since the heat energy generated for drying can be decreased.

On the other hand, the process shown in FIG. 4 in which only the high temperature heating step 3 is applied without passing through the low temperature heating step 2 is advantageous in view of shortening of the total processing time before completing the high temperature heating step 3.

However, it is appropriate to apply hot air heating at 120 to 180° C. for 2 to 8 minutes in the high temperature heating step 3 even when the low temperature heating step 2 is omitted, although the conditions are different depending on the material and quality of the clothes.

A preferable embodiment of the hanger 5 used for the conveyer will be described with reference to FIG. 5. The hanger 5 in this example is able to fold arms for attaching and detaching the clothes to enable the clothes to be promptly attached and detached. While the material for producing the hanger 5 may be appropriately selected with the proviso that it is resistant to high temperatures, stainless steel is used in this example. The hanger 5 comprises a central part 52 provided with a hook 51, and right and left arms 53 extending to both sides from the central part 52. Although the hook 51 is curved so as to be able to convey by being suspended under the conveyer chains 30 and 36a, the actual shape thereof may be arbitrarily changed so long as it is attachable and detachable to convey means such as the conveyer chains 30 and 36a. A switching means for opening and closing the right and left arms 53 is provided at the central part. In more detail, the central part 52 comprises right and left grips 54 rotatably attached at the base of the hook 51 so that the upper end of the arm is rotatable, and the right and left grips 54 are connected to the base side of the arms 53 so as to be able to slidably rotate. The ends of the right and left arms 53 are rotatably connected to a connection member 55, and a link is formed with the right and left grips 54, right and left arms 53, and connection member 55. The right and left arms 53 are closed by gripping the right and left grips 54 as shown in FIG. 5(B) so that the grips come close with each other. An elastic body such as a linear spring 56 is provided between the right and left grips 54 so that the grips 54 are usually biased in the direction for leaving with each other. The right and left arms 53 are made to open with the spring.

The construction as shown in FIG. 6 is employed in the connection member 55 so as to be able to reliably maintain the open state of the arms 53. While connection axes 57 are provided at the connection member 55 for connecting between the arms 53, these connection axes are unable to rotate when the arms 53 are open, and are rotatable when the arms 53 are closed. In more detail, while an axis insertion holes 58 for inserting each rotation axes 57 is provided at each arm 53, a rotation unable part 58a and rotatable part 58b are adjoining in the insertion hole 58. The rotation unable part 58a makes the connection axis 57 to be unable to rotate by being engaged with the connection axis 57, while the rotatable part 58b permits the connection axis 57 to rotate without engaging with the connection axis 57. The cross section of the connection axis 57 is rectangular in the drawing, and the rotation unable part 58a is also rectangular to make the connection axis unable to rotate. On the other hand, the rotatable part 58b is circular with a larger size than the connection axis 57 to permit the connection axis to rotate. However, the shape of the insertion hole is not restricted above, and may be appropriately changed. For example, the cross section of the connection axis 57 is made to be a partly cut-off circle for allowing the connection axis to be unable to rotate by providing a projection for engaging the cut-off portion at the rotation unable part 58a. On the other hand, such projection is not provided at the rotatable part 58b to permit the connection axis to rotate. The connection axis 57 is made to be unable to rotate by positioning it at the rotation unable part 58a when the arms 53 are open, by providing the rotation unable part 58a at a distal position from the rotatable part 58b. When the right and left grips 54 are made to come close with each other by gripping them, the right and left arms 53 are also made to come close with each other to allow the rotation axis 57 to displace from the rotation unable part 58a to the rotatable part 58b, thereby putting the rotation axis in a rotatable state. The arms 53 are closed with rotation by permitting the grip 54 to come close by gripping. When the gripping hand is released, the arms are spontaneously return to their original state by the action of the elastic body such as the linear spring 56, and the arms 53 are open in a rotation unable state. Accordingly, the clothes may be readily attached by releasing the hand from the grips 54 to open the right and left arms 53, after inserting the right and left arms 53 into the clothes. An inverse movement may permit the clothes to be detached. However, the hunger 5 employed may be unable to open and close without being restricted to the switching type as shown in FIGS. 5 and 6.

The method of the invention may be applied to a long size fabrics such as woven name tapes having a plurality of embroideries of names on a cloth tape. The method will be described with reference to FIGS. 7 and 8.

The woven names are formed by intermittently applying many embroideries of names on a narrow cloth tape followed by cutting respective names. While much processing steps and processing time are required for independently applying the bacteriostatic and antibacterial processing to the tape and thread, such processing may be applied on the name tape having plural embroideries of names for enabling efficient processing.

FIG. 7 illustrates the entire process of this processing method. In this example, a long size fabric such as the woven name tape having embroideries on a woven fabric are wound around a winding core m. The wound tape is drawn out to continuously subject it to the composition adhesion step 1, low temperature heating step 2 and high temperature heating step 3, followed by winding on a winding core n.

The composition adhesion step 1 comprises a primary immersion step 100, a primary wringing step 110 after the primary immersion step 100, a secondary immersion step 120 after the primary wringing step 110, and the secondary wringing step 130 after the secondary immersion step 120. In the primary immersion step 100 and secondary immersion step 120, the long size fabric t is submerged in a composition solutions filled in the primary and secondary immersion vessels 101 and 121, respectively. The long size fabric t is submerged in the composition solution by being guided with guide rollers 102 and 122. The composition used is the same as used in the foregoing embodiments.

Excess composition solution is removed by wringing with a wringing device in the primary and secondary wringing steps 110 and 130. Primary and secondary wringing rollers 111 and 131 are disposed in the vicinity of outlets of the primary and secondary immersion vessels 101 and 121, and the solution is removed by passing the long size fabric through between the wringing rollers 111 and 131 which are in compression contact with each other.

The secondary immersion and secondary wringing may be omitted. (Adding the primary and secondary wringing steps 120 and 130 are not restricted in the composition adhesion step 1 in the process shown in FIG. 1. However, such steps are not always necessary in the process shown in FIG. 1.) However, the drawn out long size fabric is not always required to be continuously processed. Instead, the drawn out long size fabric is immersed at once using the same device as in the foregoing examples, and the solution may be removed using a centrifugal dehydrator.

γ-Linolenic acid (or α-linolenic acid) and tea tree oil may be fixed on the fibers (clothes) by employing the second or third method in place of the method above.

Furthermore, lemon-scented tea tree oil may be used in place of tea tree oil in any of the methods above.

In a preferable example of the blending ratio between γ-linolenic acid and tea tree oil, the content of tea tree oil is 0.2 g, and the content of γ-linolenic acid is 0.066 g, in 100 g of the blended chemicals (composition).

When Borage oil is used for blending γ-linolenic acid, the content of Borage oil should be 0.3 g in 100 g of the blended chemical, since 0.3 g of Borage oil contains 0.066 g of γ-linolenic acid.

The proportion of the quantity of the chemicals (composition) to the quantity of the fibers (clothes) is preferably 1:1.

For example, the quantity of the chemicals used for a T-shirt for children is 100 g. Accordingly, the quantity of the chemicals used for 150 g of T-shirt is 150 g, which comprises 0.3 g of tea tree oil and 0.099 g of γ-linolenic acid.

A proportion of one third of the above blending ratio between γ-linolenic acid and tea tree oil in the chemicals is sufficient for exhibiting the effect above. Accordingly, the invention may be applied by reducing the blending ratio of γ-linolenic acid and tea tree oil from the ratio above for reducing the product cost. However, it is preferable to determine the blending ratio of γ-linolenic acid and tea tree oil in the range above for obtaining more reliable effect, and it is adequate to maintain the blending ratio above particularly for asteatosis of aged persons.

### BRIEF DESCRIPTION OF THE DRAWINGS

FIG. 1 is a block diagram showing an example of the method for producing the clothes of the invention.

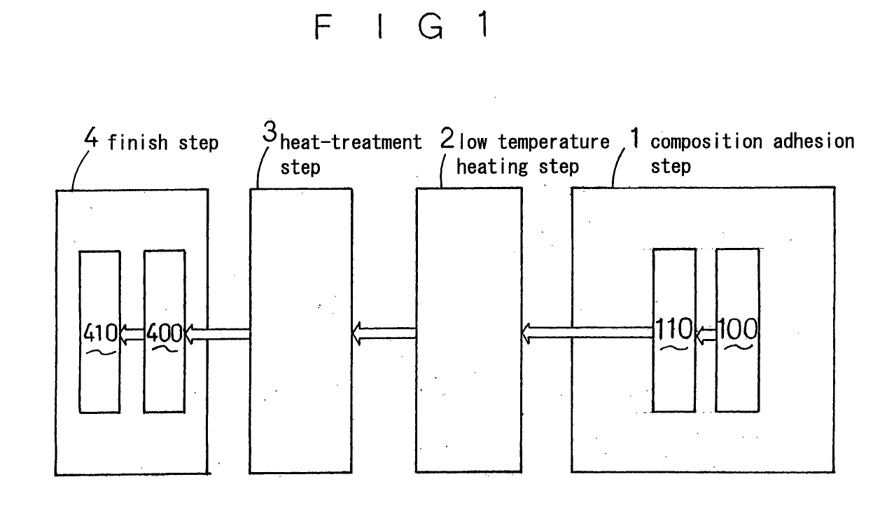

FIG. 2(A) illustrates a side view of the heating device of the invention, FIG. 2(B) illustrates a front view thereof, and FIG. 2(C) illustrates a front view thereof.

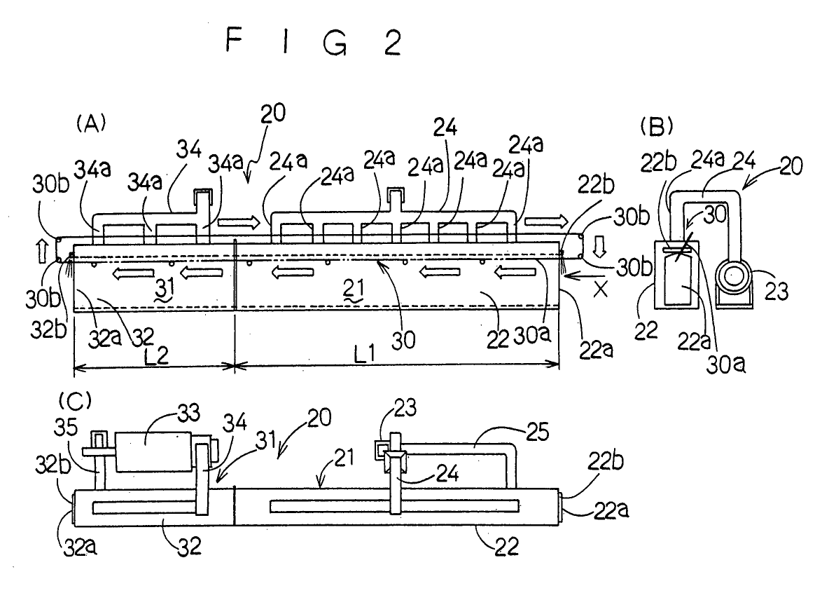

FIG. 3(A) illustrates the low temperature heating device of the heating device in the method other than the method above, and FIG. 3(B) illustrates the high temperature heating device of the heating device.

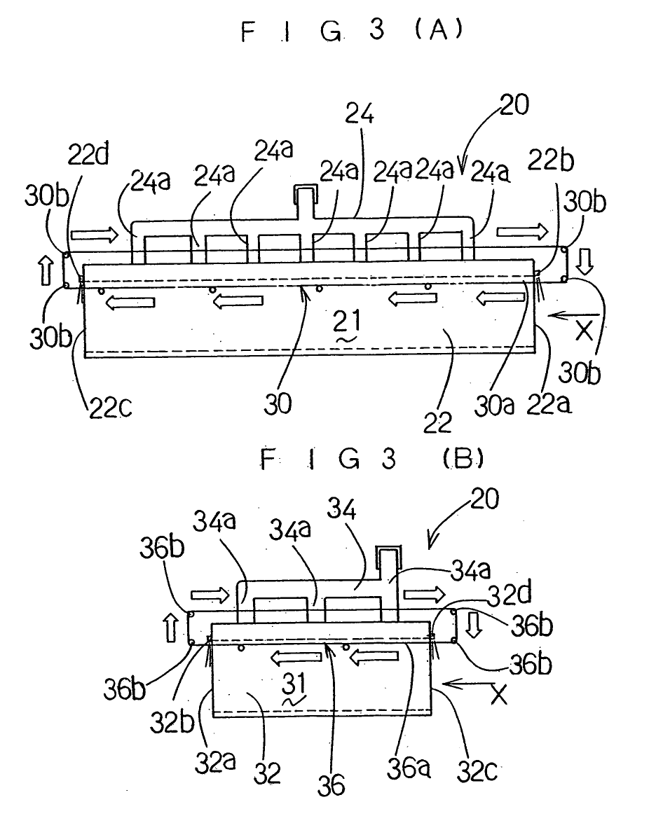

FIG. 4 is a block diagram showing another method for producing the clothes of the invention.

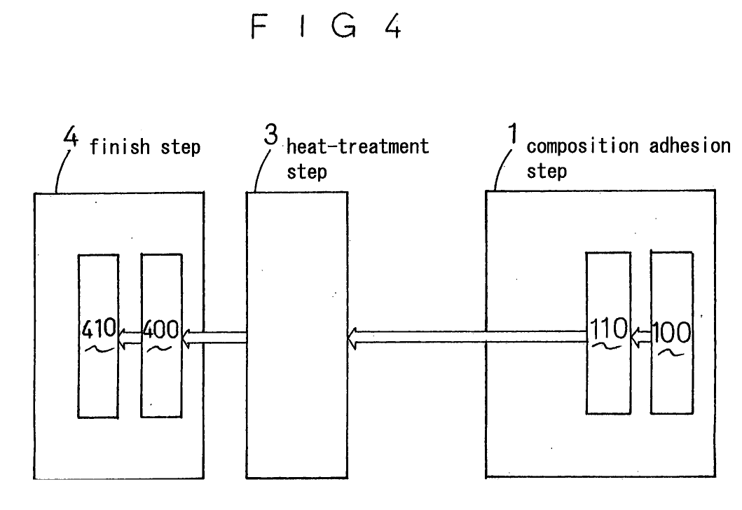

FIG. 5(A) is a front view of a hanger in an open state suitable for use in the production process of the clothes of the invention, and FIG. 5(B) is a front view in the closed state thereof.

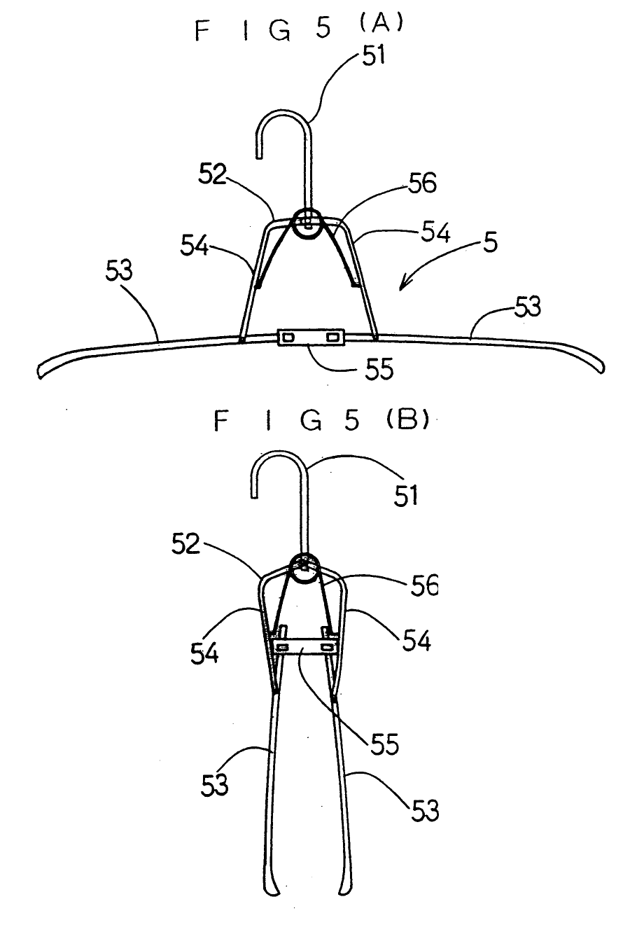

FIG. 6(A) is a plane view of the connection member of the hanger, and FIG. 6(B) shows across section along the line VI-VI in FIG. 6(A).

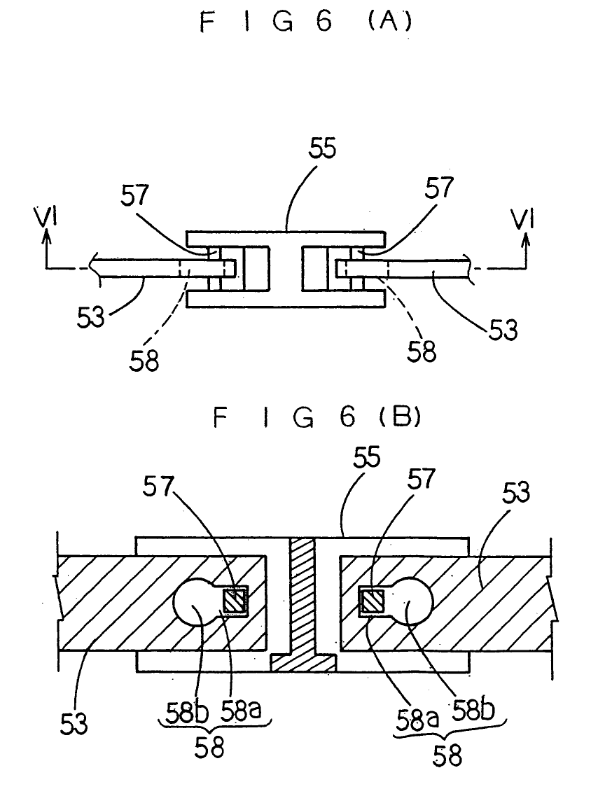

FIG. 7 illustrates the production process of a different method of the invention.

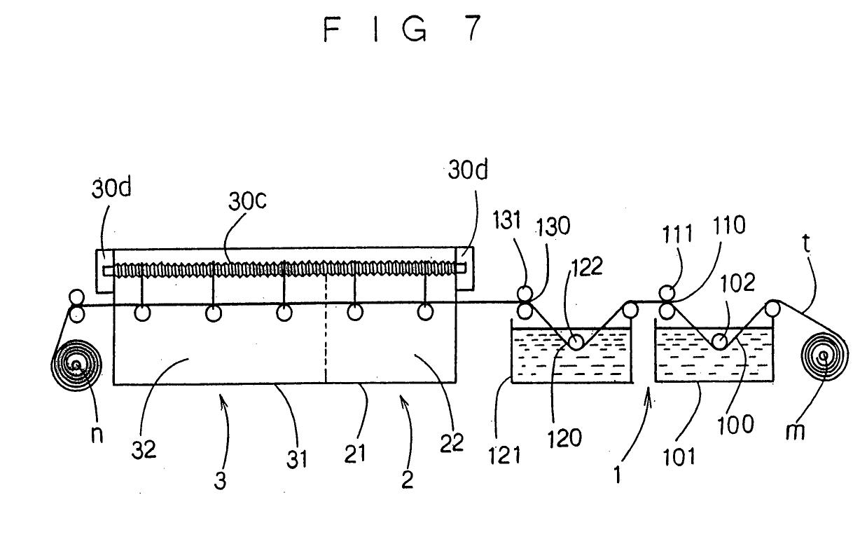

FIG. 8 is a perspective view of the supporting member of the long size fabric according to the method in FIG. 7.

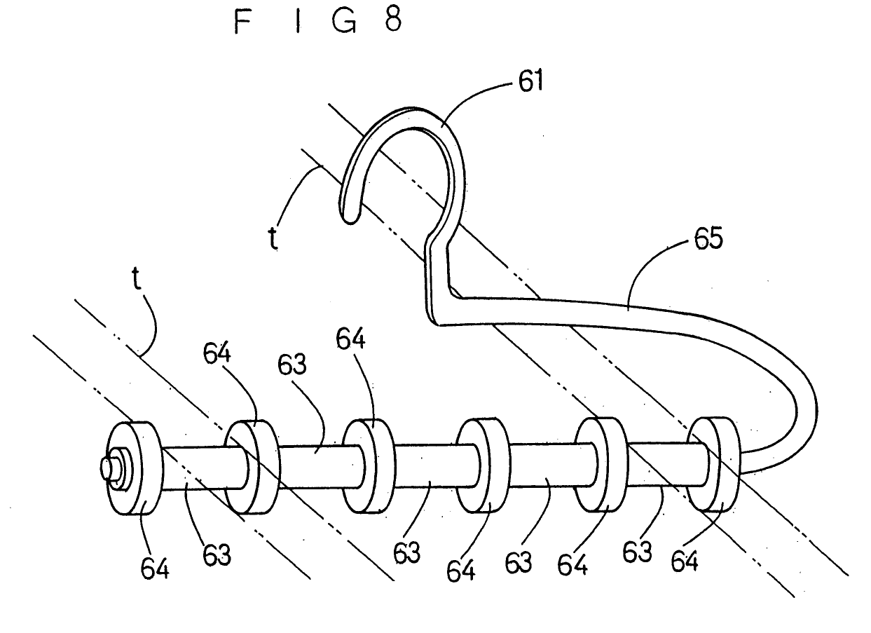

FIG. 9(A) describes the result of questionnaires with respect to rash in the example of the invention, and FIG. 9(B) described the result of questionnaires with respect to itching thereof.

FIG. 10 describes the result of questionnaires with respect to dryness in the example above.

FIG. 11 is a schematic drawing of a chemical clathrated with monochlorotriazinyl-β-cyclodextrin.

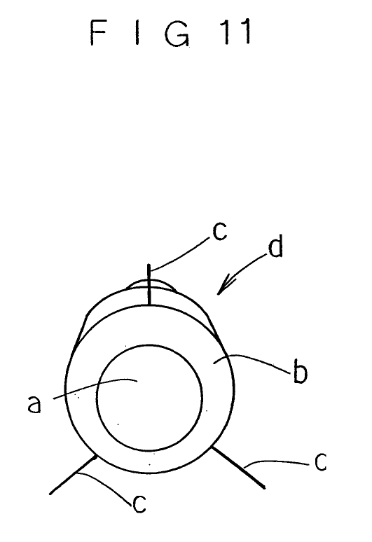

FIG. 12 shows the chemical in FIG. 11 fixed on the clothes.

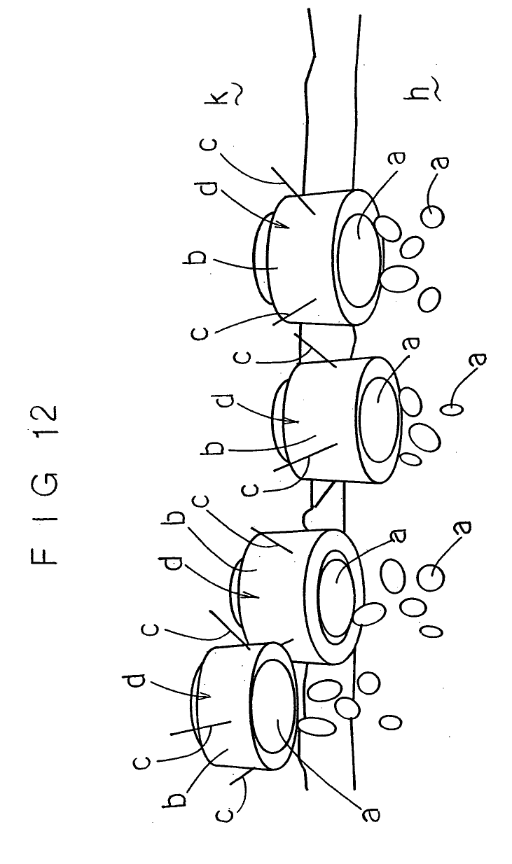

FIG. 13(A) describes a calibration curve with respect to a processed fabric B, and FIG. 13(B) describes a calibration curve with respect to a processed cloth C.

### BEST MODE FOR CARRYING OUT THE INVENTION

Thirteen normal adults were subjected to patch tests and light irradiation patch tests using a processed cloth containing γ-linolenic acid (cotton 100%, cloth before washing containing 1.06 m/g of γ-linolenic acid) in order to confirm no incidence of contact dermatitis. A skin allergy suppressing underwear for infants was produced using the cloth.

Twenty one patients (age 3 to 7) with mild or medium atopic dermatitis visiting the outpatient clinic of the Department of Dermatology, Wakayama Medical University, during the time from December 2000 to February 2001 were asked to wear the skin allergy suppressing underwear, and the results were analyzed.

This therapy was admitted by the Committee of Ethics, Wakayama Medical University.

The patients were examined by a doctor at the first medical examination, and confirmed that they are in cases (the degree of seriousness) suitable for the wearing test. The doctor obtained written consent after explaining the object and method of the treatment to the protector of the patient. The patient was asked to visit the hospital one, two and for weeks after teat wearing. The doctor in charge was observed the changes of rash, and investigated the change of the degree of rash, itching and dryness of the skin based on the questionnaires of the protector. The rate of trans-epidermal water evaporation, keratin water content and skin fat content were also measured depending on the cases.

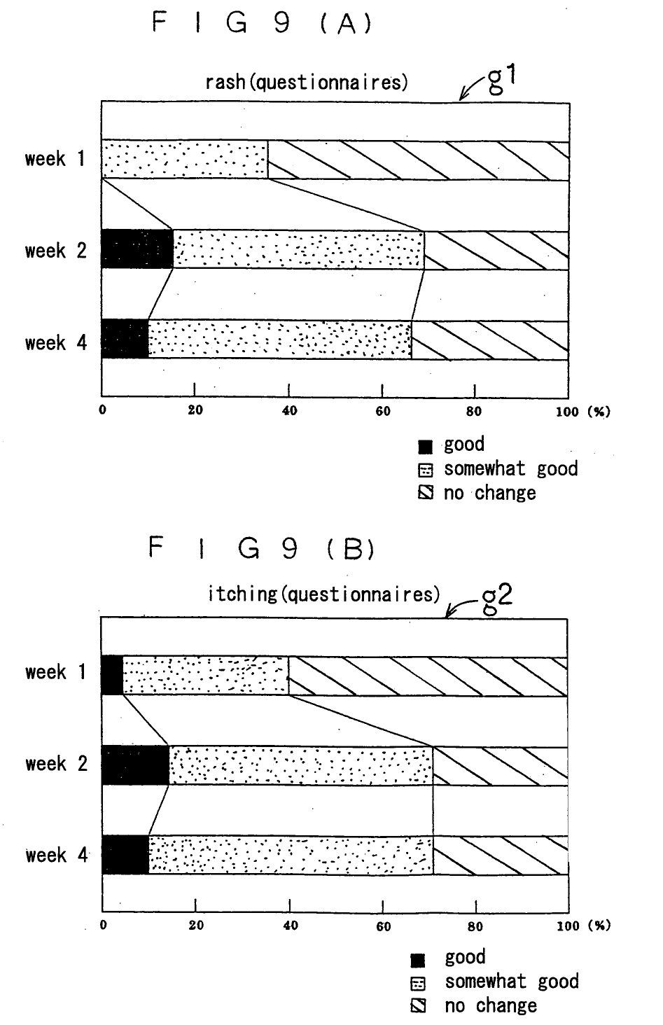

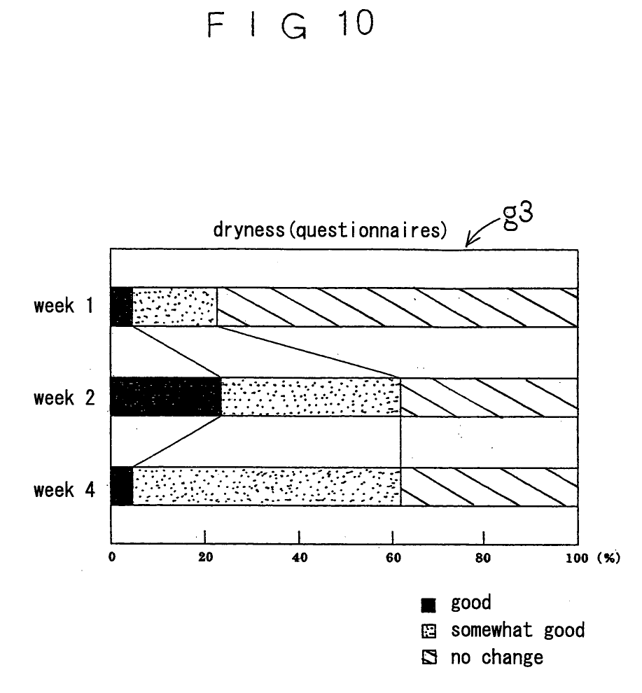

The results are shown in FIGS. 9 and 10, and the background of the patient is shown in Table 3. The components of concomitantly used medicines in Table 3 are listed in Table 4. Table 5 describes the changes of rash observed in the doctor's examination four weeks after the start of the test wearing (the percentage in the parenthesis denotes the proportion of the number of the corresponding patients to the number of the patients as the remainder of subtraction of the patients recognized to have no rash from the total number of the patients).

FIG. 9(A) is a graph (g1) showing the results of the questionnaires on rash, FIG. 9(B) is a graph (g2) showing the results of the questionnaires on itching, and FIG. 10 is a graph (g3) showing the results of the questionnaires on dryness. The dark portion shows “good” results, the spotted portion shows “somewhat good” results, and the “oblique line” portion shows “no changes”.

As shown in FIGS. 9 and 10, improvements of rash (erythema and scratch wound) are observed in about 60% of patients. From the results of the questionnaires of the protectors shown in FIGS. 9(A), 9(B) and 10, 70% or more of the patients showed some improvements of “good” and “somewhat good” in two weeks' duration of wearing test. While the improvement ratio on dryness of the skin is inferior to the improvement ratio on rash, about 60% of the patients showed “good” or “somewhat good” results.

The feeling of the protectors is almost good, saying that “the incidence of weak-up by itching was reduced”, “does not scratch” and “the skin became moist”. However, the conditions tend to be hardly improved as the degree of itching is more serious.

Accordingly, the clothes of the invention seems to be effective to patients of merely dry skin, or to patients with mild rash, if any. Although there are a few data on the trans-epidermal water evaporation rate, water content in keratin and skin fat content, it was confirmed that the remaining quantity of the medicines is reduced after 20 times of washing in the home.

An antibacterial test on Staphylococcus aureus (ATCC 6538P) was performed by a quantification assay according to JIS L1902 (standard test method; examiner: Japan Spinners Inspecting Foundation Kinki Office/1-18-15 Uemachi, Chuo-ku, Osaka city, Japan).

The results are shown in Table 6 below.

In the chemicals used for the test, 0.066 g of γ-linolenic acid and 0.044 g of tea tree oil are blended in 100 g of the chemicals.

The test results are judged to be effective when the value of log B−log A is 1.5 or more.

This test was decided to be effective since the value is 2.7 (or larger than 1.5).

The disinfection activity is defined as log A-log C, and the bacteriostatic activity is defined as log B-log C, where log C denotes the number of cells.

A cotton 100% children's underwear (cloth) having the planted cell number A and non-processed cloth cell number B was washed 60 times. Table 7 shows the number of cells, disinfection activity and bacteriostatic activity of the underwear.

Antibacterial and deodorizing processing is judged to be successful when the bacteriostatic activity is 2.2 or more. The value in the test result above is 5.8, which is sufficiently larger than this value, and the composition of the invention exhibits a quite evident effect.

Since γ-linolenic acid and tea tree oil are integrated with each other by being clathrated in the cyclic oligosaccharide, decrease of one of them may cause decrease of the other. Accordingly, the presence of bacteriostatic tea tree oil may be sensed by measuring the number of cells, disinfection activity and bacteriostatic activity, and the presence of γ-linolenic acid may be also sensed from the results above.

Accordingly, fixing abilities of both γ-linolenic acid and tea tree oil may be also confirmed from the results above.

The test data of the number of sustaining washing will be described below with respect to the processed cloth on which tea tree oil clathrated with monochlorotriazinyl-β-cyclodextrin is fixed by a covalent bond.

A cotton 100% underwear made of the cotton cloth after 100 times of washing was quantitatively tested for antibacterial property according to JIS L 0217 103 with respect to Staphylococcus Aureus ATCC 6538P (examiner: Japan Spinners Inspecting Foundation Kinki Office/1-18-15 Uemachi, Chuo-ku, Osaka city, Japan). A baby powder soap (produced by Shabondama Soap Co.) was used in this test.

The test results are shown in Table 8.

The test results are judged to be effective when the value of log B−log A is 1.5 or more.

This test was decided to be effective since the value is 2.8 (or larger than 1.5).

The disinfection activity is defined as log A-log C, and the bacteriostatic activity is defined as log B-log C, where log C denotes the number of cells.

The sample used for this test is shown in Table 9 below.

The antibacterial and deodorizing processing is judged to be successful when the bacteriostatic activity is 2.2 or larger. The test results above show the activity of 5.8 that is larger than the value above, and the composition shows a quite evident effect.

The fact that the antibacterial property of the processed cloth is high even after repeated washing of the number above show that high degree of fixing of clrolotriazinyl-β-cyclodextrin and tea tree oil on the processed cloth.

γ-Linolenic acid clathrated with clrolotriazinyl-β-cyclodextrin was fixed on the cloth by covalent bonds. This γ-linoienic acid processed cloth was subjected to washing resistance test, and the results are shown blow (examiner: Bio Research Corporation of Yokohama, Research and Development Department, Development Division, 13-46 Daikoku-cho, Yokohama, Japan).

This test was performed using processed cloth B containing 1.62 mg of fatty acids per 1 g and processed cloth G containing 8.00 mg of fatty acids per 1 g. Actually, the processed cloth as an object of comparison was Borage oil processed cloth, and γ-linolenic acid clathrated with monochlorotriazinyl-α-cyclodextrin was fixed by covalent bond to the processed cloth G for confirming the effect of the test.

The processed clothes B and G were subjected to 20 times and 30 times of washing, respectively, for preparing the test samples. Then, the content of the fatty acids in each sample was measured, and the processed clothes B and G were compared with each other with respect to resistance to washing.

In each table hereinafter, the fatty acid (Borage oil) from the processed cloth B is named as “B oil”, and the fatty acids extracted from the processed cloth G is named as “G oil”, if necessary.

For extracting the fatty acids, each of the processed cloth was cut into small pieces, and the fatty acid therein was extracted with diethyl ether by adding behenic acid as an internal standard. After concentrating the diethyl ether phase, the extract was saponified and esterified with methyl alcohol, followed by analysis by GC (gas chromatography). The content of the oil per 1 g of the processed cloth was calculated as an index of γ-linolenic acid as the assay composition.

Borage oil was extracted from the cloth by the following procedure.

After cutting 5 g each of the sample cloth into small pieces, the pieces were introduced into a 100 ml of capped conical flask. Behenic acid (5 mg) was added as the internal standard, and the flask was shaken for about 15 hours by adding 70 ml of diethyl ether. The diethyl ether phase was collected thereafter and concentrated, and the residue was saponified and esterified with methanol to prepare a sample for the GC analysis. For methylation above, the extract after concentration was placed in a screw capped test tube. Then, 6 ml of 1N KOH solution in methanol, 7 ml of BF₃ solution in methanol and 2 ml of toluene was added in the test tube, which was immersed in a boiling water bath for 5 minutes.

After cooling the test tube, 35 ml of aqueous saturated NaCl solution and 5 ml of ether were added in the test tube. After shaking and extraction, the ether phase was separated from the aqueous phase, and the ether phase was analyzed by gas chromatography after drying the ether phase with anhydrous Na₂SO₄.

A calibration curve was prepared in a separate run.

The component of the extract was analyzed under the conditions shown in Table 10. Since oil G and oil B extracted from the processed clothes G and B, respectively, are mixed oils, γ-linolenic acid (methyl ester) as the object of analysis was used as the index.

The content of the fatty acid (oil) per 1 g of each of the processed clothes B and G is shown in Table 11. The data in Table 11 show an average value obtained from respective number of washing times, as shown in Table 12 with respect to the processed cloth B and in Table 13 with respect to the processed cloth G.

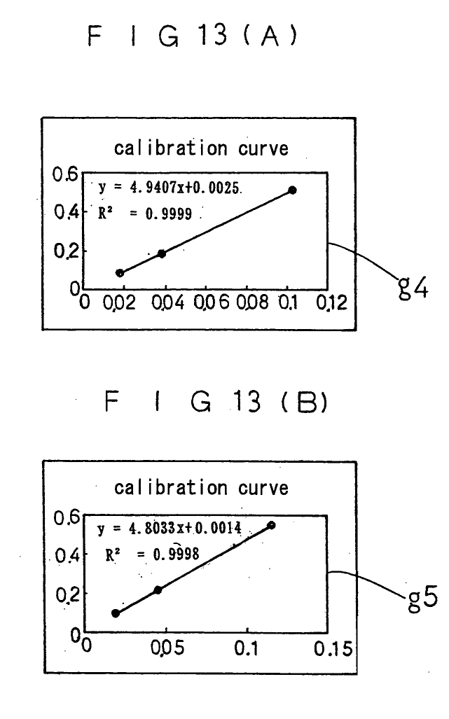

FIG. 13(A) shows the calibration curve g4 of the processed cloth B, and FIG. 13(B) shows the calibration curve g5 of the processed cloth G.

For forming each calibration curve in FIG. 13, the samples for preparing the calibration curves were prepared from G oil, B oil and behenic oil, followed by saponification and esterification with methanol. The weight ratio between B oil and behenic oil is shown in Table 14, and the area ratio between γ-linolenic acid and behenic oil is shown in Table 15. The weight ratio between G oil and behenic oil in Table 14, and the area ratio between γ-linolenic acid and behenic oil are shown in Table 16.

As shown in FIG. 10, the content of the fatty acid was higher in the processed cloth G than in the processed cloth B even after 20 ant 30 times of washing.

Although the washing number of times and was not proportional to the content of the fatty acid, the processed cloth on which γ-linolenic acid clathrated with monochlorotriazinyl-α-cyclodextrin was fixed with covalent bonds showed a higher fatty acid content. No peak of γ-linolenic acid was detected (n.d., or no data) from the analysis of the non-processed cloth O after 20 times of washing.

This means that the sample on which γ-linolenic acid clathrated with monochlorotriazinyl-β-cyclodextrin was fixed with covalent bonds has a higher fixing ratio on the cloth as compared with other samples.

TABLE 1
COMPOSITION OF TEA TREE OIL

| 1,8-cineol | 15% or less |
|---|---|
| α-terpinene | 5.0-13.0% |
| γ-teripnene | 10.0-28.0% |
| paracimene | 0.5-12.0% |
| terpinene-4-ol | 30.0% or more |
| α-terpineol | 1.5-8.0% |
| α-pinene | 1.0-6.0% |
| terpinolene | 1.5-5.0% |
| limonene | 0.5-4.0% |

TABLE 2
COMPOSITION OF LEMON-SCENTED TEA TREE OIL

| | | | | SKIN |
|---|---|---|---|---|
| | | ORAL | SKIN | SENSI- |
| COMPONENT | CONTENT | TOXICITY | IRRITATION | TIVITY |
| geranial | 30.8% | none | weak | yes |
| (citral a) | | | irritation | |
| neral (citral | 27.0% | none | weak | yes |
| b) | | | irritation | |
| citronellal | 20.0% | none | weak | yes |
| | | | irritation | |
| citronelol | 2.5% | none | weak | none |
| | | | irritation | |
| trans | 2.4% | unknown | unknown | unknown |
| isocitral | | | | |
| myrcense | 2.3% | none | weak | none |
| | | | irritation | |
| linalol | 2.1% | none | none | none |
| iso-pulegol | 2.0% | unknown | unknown | unknown |
| cis-isocitral | 1.6% | unknown | unknown | unknown |
| limonene | 0.5% | none | weak | none |
| | | | irritation | |
| α-pinen | 0.5% | weakly toxic | none | yes |
| methyl | 0.2% | none | weak | none |
| heptanone | | | irritation | |
| terpine-4-ol | 0.1% | toxic | weak | none |
| | | | irritation | |
| sabinene | 0.1% | none | unknown | unknown |
| terpinolene | 0.1% | unknown | unknown | unknown |
| β-pinen | 0.1% | none | unknown | unknown |
| α-thujene | small | unknown | unknown | unknown |
| trans-β-ocimen | small | none | none | none |

(see http://www.airgreen.co.jp/teatree/lemon.html)

TABLE 3
BACKGROUND OF THE PATIENT

| ITEM | CONTENT | NUMBER OF CASES |
|---|---|---|
| Sex | Male | 11 |
| | Female | 10 |
| Age | Age 2 | 3 |
| | Age 3 | 1 |
| | Age 4 | 4 |
| | Age 5 | 8 |
| | Age 6 | 4 |
| | Age 7 | 1 |
| Seriousness | Mild | 10 |
| | Medium | 11 |
| | Serious | 0 |
| Extent of Dry Skin | None | 0 |
| | Mild | 5 |
| | Medium | 16 |
| | Serious | 0 |
| Complication | None | 13 |
| | Yes | 8 |
| Concomitant Use | None | 1 |
| Medicine | Yes | 20 |

TABLE 4
COMPONENT OF CONCOMITANT
USE MEDICINE (TOTAL 100%)

| | |
|---|---|
| Steroid alone | 5% |
| None | 9% |
| Protopic Ointment | 9% |
| Humectant alone | 27% |
| Steroid + Humectant | 45% |
| Unknown | 5% |

TABLE 5
DEGREE OF IMPROVEMENT OF RASH

| | Improved | No Change | Worsened | No Observation |
|---|---|---|---|---|
| Erythema | 10 | 5 | 0 | 6 |
| | (66.7%) | (33.3%) | (0%) | |
| Trace of | 12 | 7 | 0 | 2 |
| Scratch | (63.2%) | (36.8%) | (0%) | |

TABLE 6

| | | |
|---|---|---|
| Planted Cell Number [A] | 2.4 × 10⁴ | 10 gA 4.4 |
| Non-processed Cloth Cell Number | 1.4 × 10⁷ | 10 gB 7.1 |
| [B] | | |

(standard cotton cloth is used for non-processed cloth)

TABLE 7

| | Cell Number | Disinfection | Bacteriostatic |
|---|---|---|---|
| Sample | log C | Activity | Activity |
| Cotton 100% | 1.3 | 3.1 | 5.8 |
| children's | | | |
| underwear | | | |

(A, B: 60 times of washing)

TABLE 8

| | | |
|---|---|---|
| Planted Cell Number [A] | 1.8 × 10⁴ | 10 gA 4.3 |
| Non-processed Cloth Cell Number | 1.3 × 10⁷ | 10 gB 7.1 |
| [B] | | |

(standard cotton cloth is used for non-processed cloth)

TABLE 9

| | Cell | | |
|---|---|---|---|
| | Number | Disinfection | Bacteriostatic |
| Sample | log C | Activity | Activity |
| Cotton 100% | 1.3 | 3.0 | 5.8 |
| Underwear | | | |

TABLE 10
GC analysis conditions

| | |
|---|---|
| Column | TC-1 (0.25 mm × 60 m) |
| Column Temperature | 240° C. |
| Injection Port | 300° C. |
| Temperature | |
| Discharger Temperature | 300° C. |
| Detector | FID |
| Carrier Gas | He 237.8 mL/min (400.2 KPa) |
| Hydrogen Gas | 47.0 mL/min |
| Air | 400 mL/min |
| Sample Injection Volume | 1 μl |
| Retention Time | γ-linolenic acid (methyl ester) 5.98 min |
| | behenic acid (internal standard) |
| | 14.59 min |

TABLE 11
Content of Fatty Acid (Oil) per 1 g of Cloth

| Washing Times | 0 time | 20 times | 30 times |
|---|---|---|---|
| B | 1.62 mg | 6.98 mg | 6.99 mg |
| G | 8.00 mg | 45.3 mg | 38.0 mg |
| Non-processed | | n.d. | |
| Cloth | | | |

TABLE 12

| | Weight | | Area | | | | | | |
|---|---|---|---|---|---|---|---|---|---|
| | Cloth | Behenic | γ-Linolenic | Behenic | | | | | |
| Sample | (g) | Acid (mg) | Acid | Acid | Area ratio | 1 | 2 | 3 | 4 |
| B-0 time-1 | 5.0122 | 5.32 | 26443 | 86557 | 0.30549811 | 1.51187452 | 8.04317243 | 1.604718972 | 1.6170289 |
| B-0 time-2 | 5.019 | 5.18 | 25236 | 79104 | 0.31902306 | 1.57869722 | 8.17765162 | 1.629338836 | |
| B-20 | 5.011 | 5.18 | 6567 | 4314 | 1.52225313 | 7.52349604 | 38.9717095 | 7.77723198 | 6.9806014 |
| times-1 | | | | | | | | | |
| B-20 | 5.0133 | 4.97 | 6579 | 5213 | 1.26203721 | 6.23784727 | 31.0021009 | 6.18397082 | |
| times-2 | | | | | | | | | |
| B-30 | 5.0129 | 5.18 | 5682 | 4089 | 1.3895818 | 6.86800682 | 35.5762753 | 7.09694495 | 6.98785417 |
| times-1 | | | | | | | | | |
| B-30 | 5.0344 | 4.97 | 6330 | 4490 | 1.40979955 | 6.96789666 | 34.6304464 | 6.87876339 | |
| times-2 | | | | | | | | | |
| 0-5 times-2 | 5.0017 | 5.18 | — | 14217 | #VALUE! | #VALUE! | #VALUE! | #VALUE! | #VALUE! |
| 0-20 | 5.03 | 4.97 | — | 6986 | #VALUE! | #VALUE! | #VALUE! | #VALUE! | |
| times-2 | | | | | | | | | |

1 Weight Ratio From Calibration Curve
2 Content of B Oil (mg)
3 Content in 1 g of Cloth (mg)
4 Average

TABLE 13

| | Weight | | Area | | | | | | |
|---|---|---|---|---|---|---|---|---|---|
| | Cloth | Behenic | γ-Linolenic | Behenic | | | | | |
| Sample | (g) | Acid (mg) | Acid | Acid | Area ratio | 1 | 2 | 3 | 4 |
| G-0 time-1 | 5.0182 | 5.32 | 164135 | 97664 | 1.68060903 | 8.07386934 | 42.9529849 | 8.559440613 | 7.9992889 |
| G-0 time-2 | 5.0103 | 5.18 | 134021 | 89483 | 1.49772583 | 7.19542646 | 37.272309 | 7.439137185 | |
| G-20 | 5.0046 | 5.32 | 55451 | 6562 | 8.45032002 | 40.5908222 | 215.943174 | 43.1489378 | 45.2530491 |
| times-1 | | | | | | | | | |
| G-20 | 5.0052 | 4.97 | 43250 | 4356 | 9.92883379 | 47.6925674 | 237.03206 | 47.3571605 | |
| times-2 | | | | | | | | | |
| G-30 | 5.0267 | 5.18 | 34472 | 4396 | 7.84167425 | 37.6673139 | 195.116686 | 38.8160595 | 37.983774 |
| times-1 | | | | | | | | | |
| G-30 | 5.0053 | 4.97 | 33961 | 4360 | 7.78922018 | 37.4153613 | 185.954346 | 37.1514886 | |
| times-2 | | | | | | | | | |
| 0-5 times-2 | 5.0192 | 5.32 | — | 12897 | #VALUE! | #VALUE! | #VALUE! | #VALUE! | #VALUE! |
| 0-20 | 5.0192 | 5.32 | — | 10929 | #VALUE! | #VALUE! | #VALUE! | #VALUE! | |
| times-2 | | | | | | | | | |

1 Weight Ratio From Calibration Curve
2 Content of G Oil (mg)
3 Content in 1 g of Cloth (mg)
4 Average

TABLE 14

| G | | | B | | |
|---|---|---|---|---|---|
| | | Behenic | | | Behenic |
| | G Oil | Acid | | B Oil | Acid |
| Std. 1 | 1.05 mg | 10.6 mg | Std. 1 | 0.99 mg | 10.5 mg |
| 2 | 2.10 mg | 9.7 mg | 2 | 1.98 mg | 10.4 mg |
| 3 | 6.25 mg | 9.4 mg | 3 | 4.95 mg | 9.6 mg |

TABLE 15

| | Weight | | | Area | | |
|---|---|---|---|---|---|---|
| | | Behenic Acid | Weight | γ-Linolenic | Behenic | |
| | B Oil (mg) | (mg) | Ratio | Acid | Acid | Area Ratio |
| Std. 1 | 0.99 | 10.5 | 0.09428571 | 4414 | 243026 | 0.01816267 |
| Std. 2 | 1.98 | 10.4 | 0.19038462 | 8043 | 208611 | 0.03855501 |
| Std. 3 | 4.95 | 9.6 | 0.515625 | 15957 | 153845 | 0.10372128 |

TABLE 16

| | Weight | | | Area | | |
|---|---|---|---|---|---|---|
| | | Behenic Acid | Weight | γ-Linolenic | Behenic | |
| | G Oil (mg) | (mg) | Ratio | Acid | Acid | Area Ratio |
| Std. 1 | 1.05 | 10.6 | 0.0990566 | 4016 | 203434 | 0.01974105 |
| Std. 2 | 2.1 | 9.7 | 0.21649485 | 8375 | 183623 | 0.04560975 |
| Std. 3 | 5.25 | 9.4 | 0.55851064 | 20499 | 177063 | 0.11577235 |

STRUCTURE 1

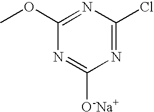

1. A skin condition-improving composition containing any one of γ-linolenic acid and α-linolenic acid.

2. A skin condition-improving composition containing a bacteriostatic agent and any one of γ-linolenic acid and α-linolenic acid.

3. A skin condition-improving composition containing any one of terpinene-4-ol, citral and citronellal, and any one of γ-linolenic acid and α-linolenic acid.

4. Clothes comprising at least any one of γ-linolenic acid, α-linolenic acid, tea tree oil and lemon-scented tea tree oil fixed thereon.

5. Clothes comprising any one of γ-linolenic acid, α-linolenic acid, tea tree oil and lemon-scented tea tree oil fixed thereon as a clathrate compound with monochlorotriazinyl-β-cyclodextrin.

6. A skin condition-improving composition comprising any one of γ-linolenic acid and α-linolenic acid, and a bacteriostatic agent, at least one of them being clathrated with monochlorotriazinyl-β-cyclodextrin.
